(19) United States
(12) Patent Application Publication
(10) Pub. No.: US 2012/0116328 A1
(43) Pub. Date: May 10, 2012

(54) Valve for urine bag

(76) Inventors: Chien-Hsiung Hung, Taipei (TW); Tzu-Chen Huang, Taipei City (TW)

(21) Appl. No.: 12/926,287

(22) Filed: Nov. 8, 2010

(51) Int. Cl. A61F 5/44

(52) U.S. Cl. 604/317

(57) ABSTRACT

A valve for a urine bag includes a guide member having a connection end at one end thereof and a plurality of holes are defined through a wall of the guide member. A sleeve is connected to the connection end and a movable member is movably located in the guide member. The movable member includes a base and a guide rod extends from an end of the base. The valve is connected to the connection part of the urine bag and when urine passes through the guide member, the movable member is pushed away from the sleeve to allow the urine to flow into the urine bag. The movable member is moved to contact the sleeve when the urine in the urine bag flows backward. The base seals the sleeve to stop the urine flowing back to the user.

### BACKGROUND OF THE INVENTION

(1) Field of the Invention The present invention relates to a valve for a urine bag, and more particularly, to a valve connected to the urine bag and prevents the urine from flows back to the user.

(2) Description of the Prior Art

A conventional urine bag 1 is shown in FIG. 1 and generally includes a connection part 11 connected to one side of the urine bag 1 and a hose 12 is connected to the connection part 11. The hose has the other end connected to the user's body so as to guide the user's urine to the urine bag 1 via the hose 12. The urine can be collected or disposed when the urine bag 1 is full. However, there is no valve connected to the urine bag 1 to prevent the urine in the urine bag 1 from flowing back to the user. Therefore, the urine bag 1 cannot be tilt or up-side-down, the connection part 11 has to be located upright, the urine bag 1 has to be located beneath the user's urinary bladder, or the urine could flow back to the user and causes other problems.

The present invention intends to provide a valve for a urine bag and the valve effectively prevents the urine from flowing back to the user so as to improve the problems that the conventional urine bags have.

### SUMMARY OF THE INVENTION

The present invention relates to a valve for a urine bag and comprises a guide member having a connection end at one end thereof and a plurality of holes are defined through a wall of the guide member. A sleeve is connected to the connection end and a movable member is movably located in the guide member. The movable member includes a base and a guide rod extends from an end of the base. When urine passes through the guide member, the movable member is pushed away from the sleeve to allow the urine to flow into the urine bag. The movable member is moved to contact the sleeve when the urine in the urine bag flows backward. The base seals the sleeve to stop the urine flowing back to the user.

The primary object of the present invention is to provide a valve for a urine bag and the valve prevents the urine in the urine bag from flowing back to the user's body.

Another object of the present invention is to provide a valve for a urine bag wherein the valve can be easily installed to the existed urine bags.

The present invention will become more obvious from the following description when taken in connection with the accompanying drawings which show, for purposes of illustration only, a preferred embodiment in accordance with the present invention.

### BRIEF DESCRIPTION OF THE DRAWINGS

FIG. 1 shows the conventional urine bag;

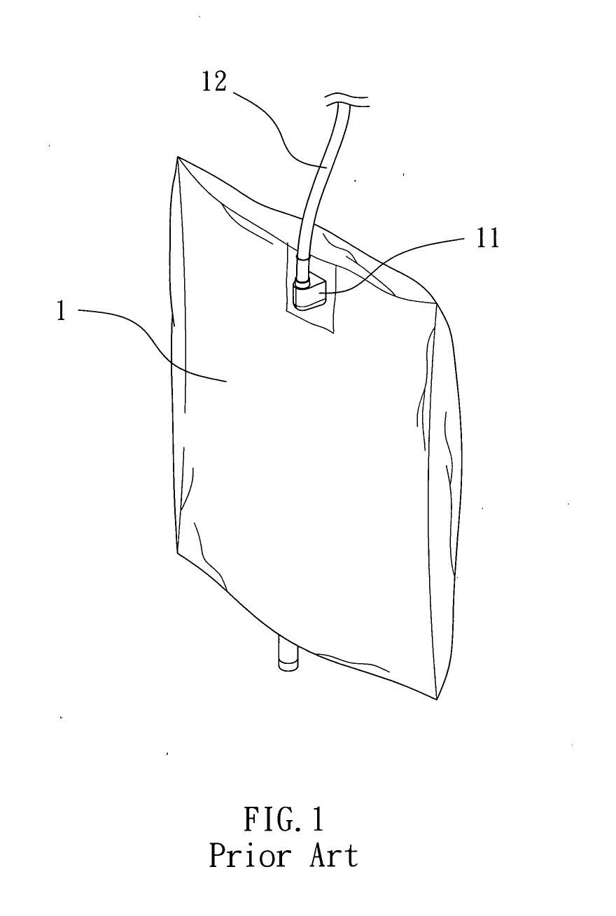

FIG. 2 is a perspective view to show the valve of the present invention;

FIG. 3 is an exploded view to show the valve of the present invention;

FIG. 4 is a cross sectional view of the valve of the present invention;

FIG. 5 is a cross sectional view to show that the hose and connection tube are to be connected to the connection part of the urine bag;

FIG. 6 is a cross sectional view to show that the valve of the present invention is to be connected to the connection tube of the urine bag;

FIG. 7 is a cross sectional view to show that the valve of the present invention is connected to the connection tube which is engaged with the connection part of the urine bag;

FIG. 8 shows that the urine flows into the urine bag;

FIG. 9 is a cross sectional view to show the urine pushes the movable member and flows into the urine bag;

FIG. 10 shows the urine bag is full, and

FIG. 11 is a cross sectional view to show the urine pushes the movable member to seal the hose.

### DETAILED DESCRIPTION OF THE PREFERRED EMBODIMENT

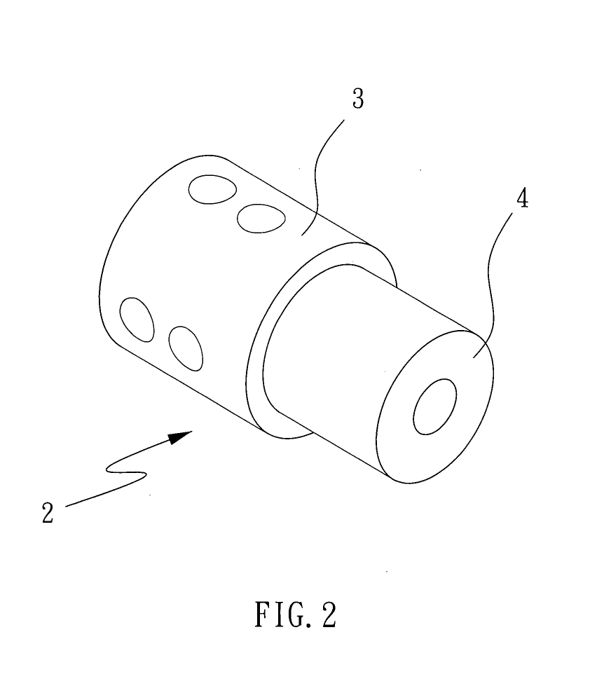

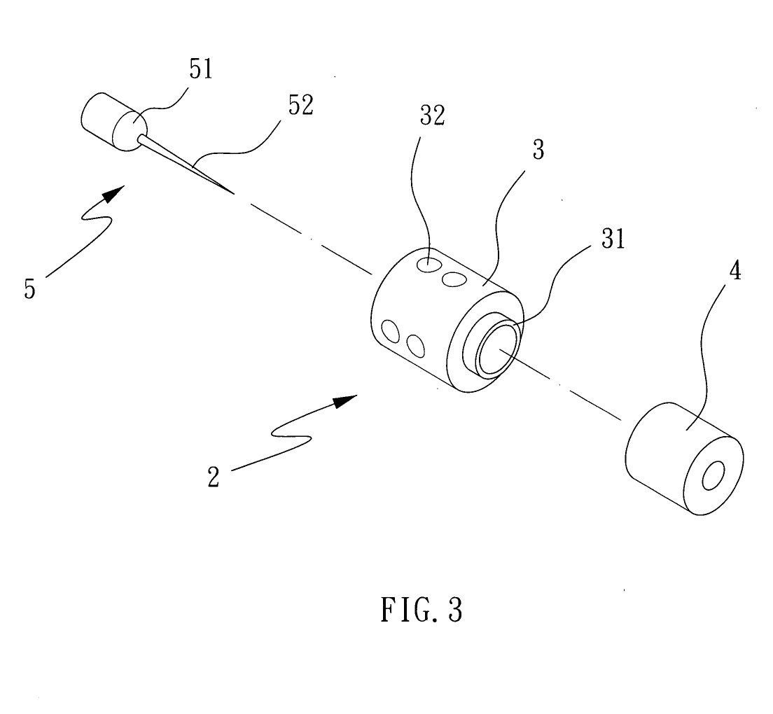

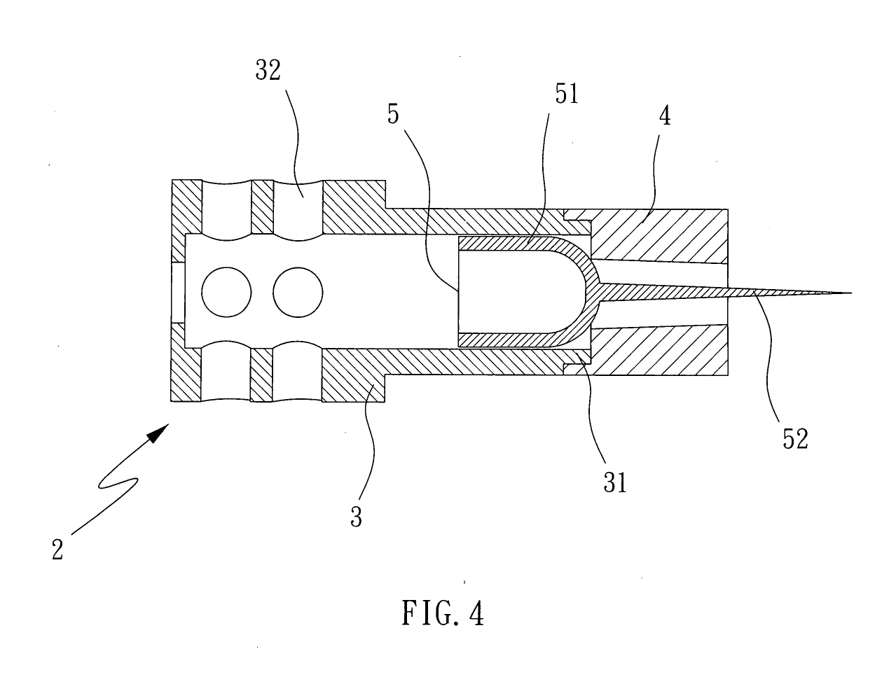

Referring to FIGS. 2 to 4, the valve 2 for a urine bag 6 of the present invention comprises a guide member 3 having a connection end 31 at one end thereof and a plurality of holes 32 are defined through a wall of the guide member 3. A sleeve 4 made of soft material is connected to the connection end 31. A movable member 5 is movably located in the guide member 3 and has a base 51 and a guide rod 52 which extends from an end of the base 51. The end of the base 51 where the guide rod 52 extends is a semi-spherical surface so as to be able to seal the sleeve 4 when the base 51 contacts the sleeve 4 as shown in FIG. 4.

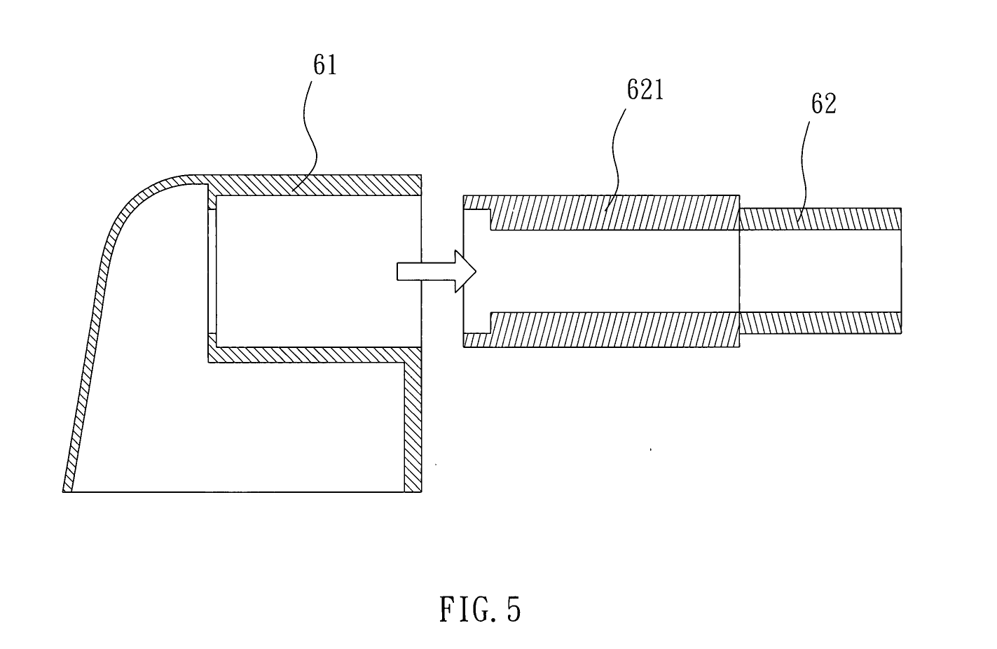

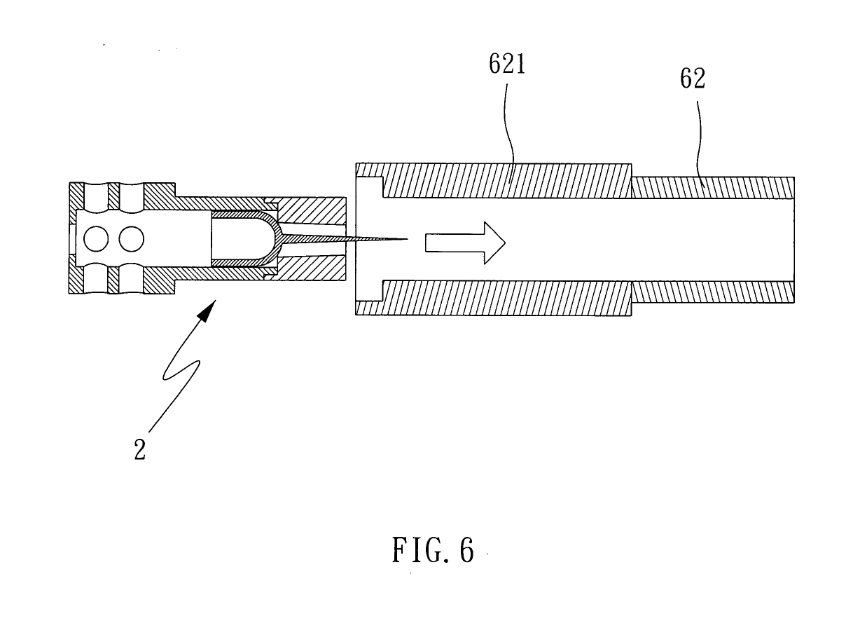

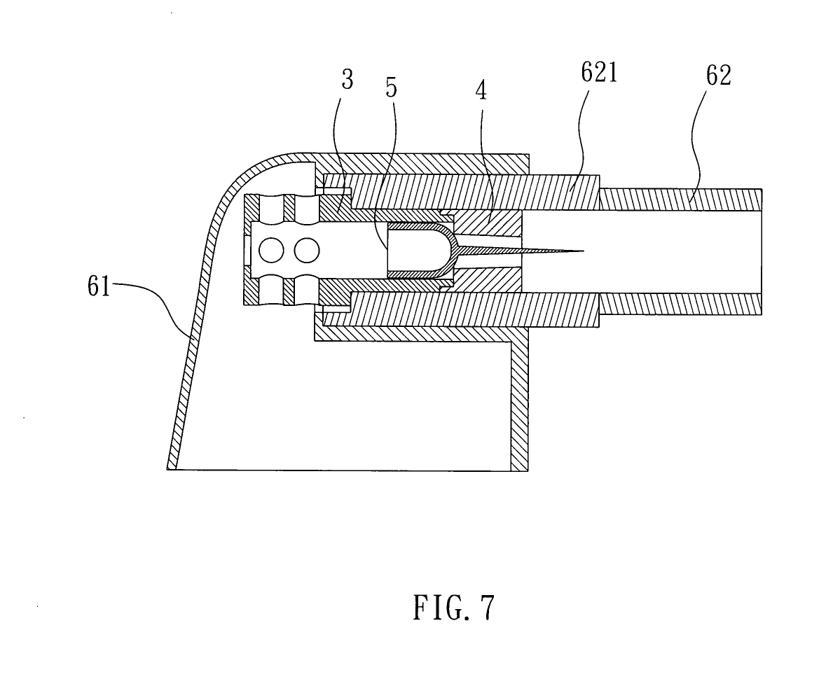

As shown in FIGS. 5 to 7, the urine bag 6 includes a connection part 61 and a connection tube 621 is securely engaged with the connection part 61. The connection tube 621 has a hose 62 connected thereto which is connected to the user. The valve 2 is inserted into the connection tube 621 as shown in FIG. 7, wherein the connection tube 621 has a shoulder to which the guide member 3 is stopped.

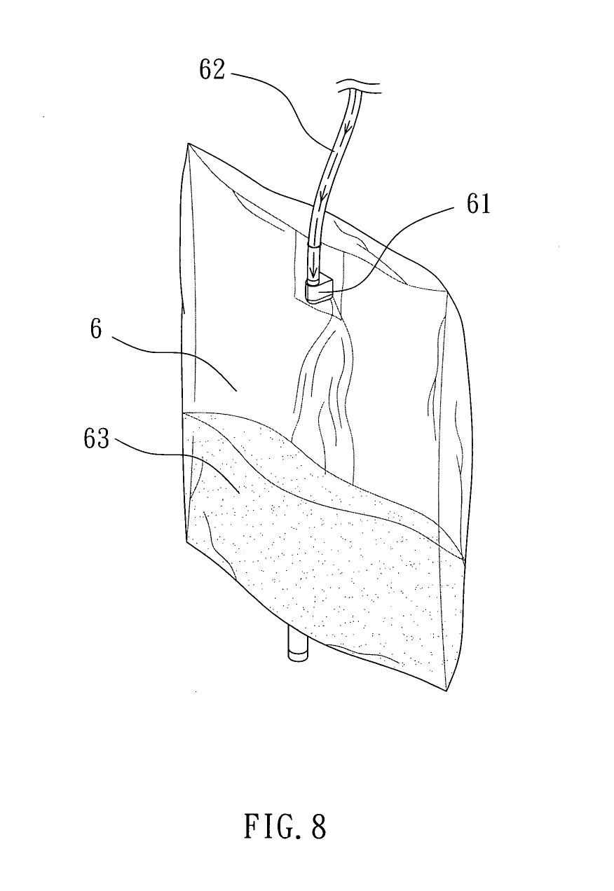

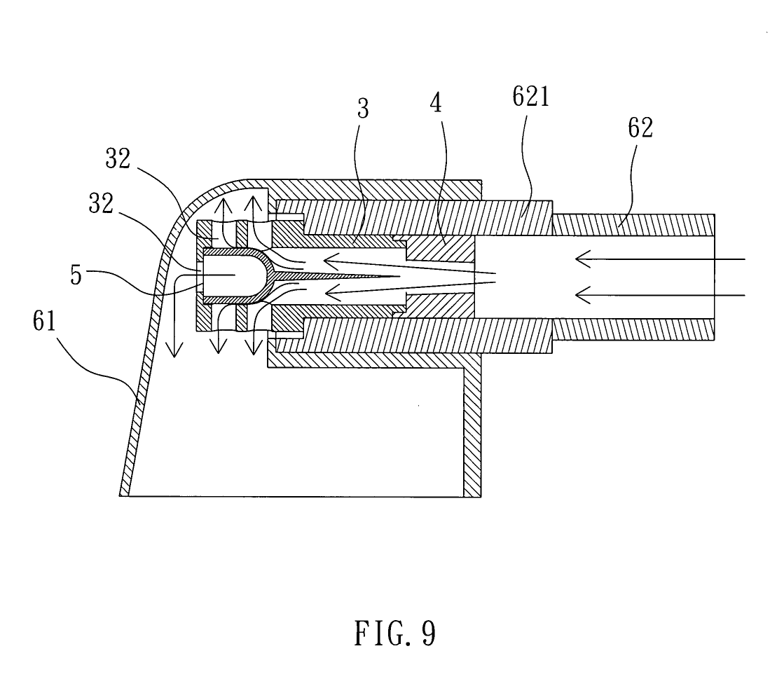

As shown in FIGS. 8 and 9, when the urine 63 from the user flows through the valve 2 via the hose 62, the pressure of the urine 63 pushes the base 51 away from the sleeve 4 so that the urine is allowed to flow through a gap between the end of the base 51 and the inside of the guide member 3 and the holes 32 of the guide member 3 and is collected in the urine bag 6.

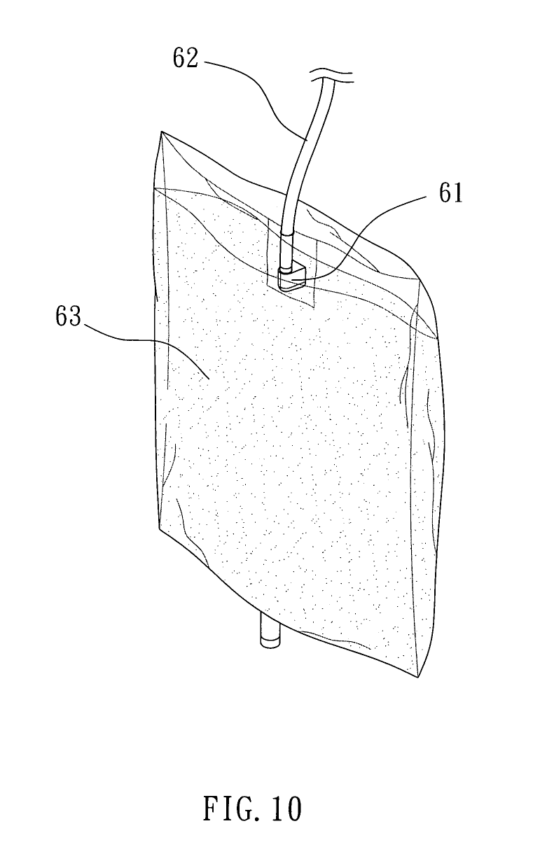

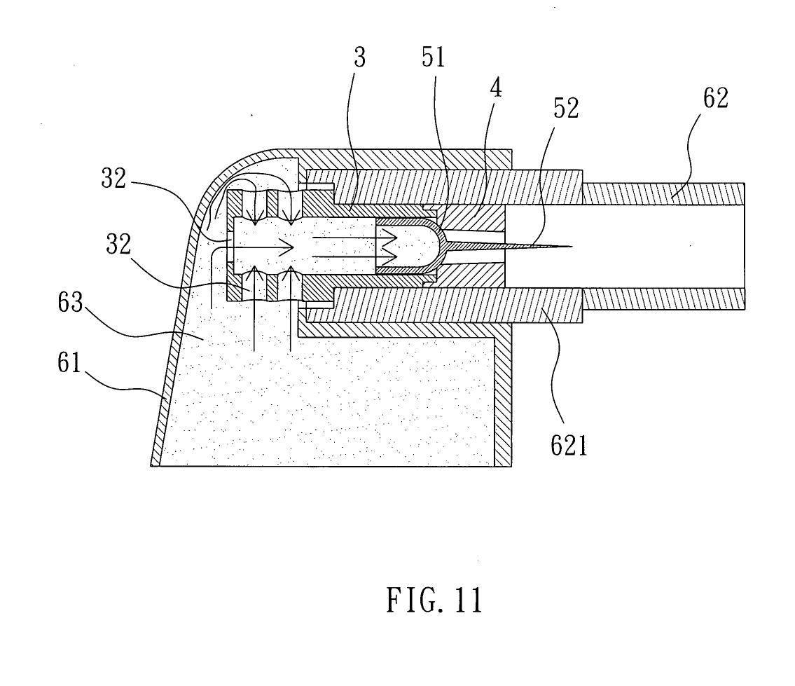

As shown FIGS. 10 and 11, when urine bag 6 is full or tilt, the urine 63 passes through the guide member 5 via the holes 32 and the base 51 is moved to contact the sleeve 4 and seals the sleeve 4, such that the urine in the urine bag 1 cannot flow backward.

It is noted that the guide rod 52 is located in the sleeve 4 when the base 51 contacts the sleeve 4 and the guide rod 52 effectively guide the base 51 to move as desired to ensure the function.

The valve 2 of the present invention can be connected to the connection tube 621 of the existed urine bag 6 without extra parts or machining process.

While we have shown and described the embodiment in accordance with the present invention, it should be clear to those skilled in the art that further embodiments may be made without departing from the scope of the present invention.

What is claimed is:

1. A valve for a urine bag, comprising:
a guide member having a connection end at one end thereof and a plurality of holes defined through a wall of the guide member;
a sleeve connected to the connection end, and
a movable member movably located in the guide member and having a base and a guide rod which extends from an end of the base, the base being pushed away from the sleeve when urine passes through the guide member, the base being moved to contact the sleeve when the urine in the urine bag flows backward.

2. The valve as claimed in claim 1, wherein the sleeve is a soft sleeve.

3. The valve as claimed in claim 1, wherein the end of the base where the guide rod extends is a semi-spherical surface.

4. The valve as claimed in claim 1, wherein the guide rod is located in the sleeve when the movable member contacts the sleeve.
